(19) United States
(12) Patent Application Publication
(10) Pub. No.: US 2006/0130245 A1
(43) Pub. Date: Jun. 22, 2006

(54) Composition for the oxidation dyeing of keratin fibres and dyeing process using this composition

(75) Inventor: Marie-Pascale Audousset, Asnieres (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'Oreal S.A.

(21) Appl. No.: 11/289,629

(22) Filed: Nov. 30, 2005

Related U.S. Application Data
(63) Continuation of application No. 09/443,506, filed on Nov. 19, 1999, now abandoned.

(30) Foreign Application Priority Data
Nov. 20, 1998 (FR) 98 14652

(51) Int. Cl. A61K 8/00

(52) U.S. Cl. 8/405

(57) ABSTRACT

The invention relates to a composition for the oxidation dyeing of keratin fibres, and in particular human keratin fibres such as the hair, comprising, in a medium which is suitable for dyeing, at least one oxidation base, 1,3-bis(β-hydroxyethyl)amino-2-methylbenzene and/or at least one of the addition salts thereof with an acid as first coupler, and at least one substituted meta-aminophenol selected as second coupler, as well as to the dyeing process using this composition.

The invention relates to a composition for the oxidation dyeing of keratin fibres, and in particular human keratin fibres such as the hair, comprising, in a medium which is suitable for dyeing, at least one oxidation base, 1,3-bis(β-hydroxyethyl)amino-2-methylbenzene and/or at least one of the addition salts thereof with an acid as first coupler, and at least one substituted meta-aminophenol selected as second coupler, as well as to the dyeing process using this composition.

It is known practice to dye keratin fibres, and in particular human hair, with dye compositions containing oxidation dye precursors, in particular ortho- or para-phenylenediamines, ortho- or para-aminophenols and heterocyclic bases, which are generally referred to as oxidation bases. Oxidation dye precursors, or oxidation bases, are colourless or weakly coloured compounds which, when combined with oxidizing products, can give rise to coloured compounds and dyes by a process of oxidative condensation.

It is also known that the shades obtained with these oxidation bases can be varied by combining them with couplers or coloration modifiers, the latter being chosen in particular from aromatic meta-diamines, meta-aminophenols, meta-diphenols and certain heterocyclic compounds.

The variety of molecules used as oxidation bases and couplers allows a wide range of colours to be obtained.

The so-called “permanent” coloration obtained by means of these oxidation dyes must moreover satisfy a certain number of requirements. Thus, it must be able to give shades of the desired intensity and it must be able to withstand external agents (light, bad weather, washing, permanent-waving, perspiration, rubbing).

The dyes must also be able to cover white hair and, lastly, they must be as unselective as possible, i.e. they must give the smallest possible coloration differences along the length of the same keratin fibre, which may in fact be differently sensitized (i.e. damaged) between its tip and its root.

Compositions for the oxidation dyeing of keratin fibres containing one or more oxidation bases, one or more 2,6-diaminotoluene derivatives as coupler, and optionally one or more additional couplers chosen from the couplers conventionally used in the field of oxidation dyeing, have already been proposed, in particular in patent applications FR-A-2,681,860 and DE-A-19,637,371, such as meta-aminophenol and 2-methyl-5-aminophenol. However, although the colorations obtained using such compositions are highly chromatic, they are not entirely satisfactory, in particular as regards their selectivity and their fastness with respect to the various treatments and natural attacking factors to which keratin fibres may be subjected.

The Applicant has now discovered that it is possible to obtain novel dyes which are capable of giving intense and highly chromatic colorations, and which show good resistance to the various attacking factors to which the fibres may be subjected, by combining at least one oxidation base, 1,3-bis(β-hydroxyethyl)amino-2-methylbenzene and/or at least one of the addition salts thereof with an acid as first coupler, and at least one suitably selected substituted meta-aminophenol as second coupler.

This discovery forms the basis of the present invention.

A first subject of the invention is thus a composition for the oxidation dyeing of keratin fibres, and in particular human keratin fibres such as the hair, characterized in that it comprises, in a medium which is suitable for dyeing:
at least one oxidation base,
1,3-bis(β-hydroxyethyl)amino-2-methylbenzene and/or at least one of the addition salts thereof with an acid, as first coupler;
and at least one second coupler chosen from the substituted meta-aminophenols of formulae (I) and (Ia) below, and the addition salts thereof with an acid: 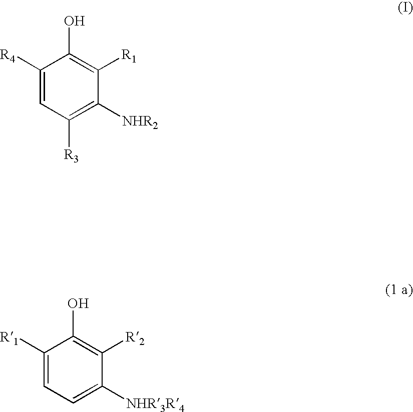
   in which:
R¹ represents a hydrogen atom, a C₁-C₄ alkyl radical, a C₁-C₄ monohydroxyalkyl radical or a C₂-C₄ polyhydroxyalkyl radical;
R₂ represents a hydrogen atom, a C₁-C₄ alkyl radical, a C₁-C₄ monohydroxyalkyl radical or a C₂-C₄ polyhydroxyalkyl radical;
R₃ represents a hydrogen atom, a C₁-C₄ alkyl radical, a C₁-C₄ alkoxy radical or a halogen atom chosen from chlorine, bromine, iodine and fluorine;
R₄ represents a hydrogen atom, a C₁-C₄ alkyl radical, a C₁-C₄ alkoxy radical, a C₁-C₄ monohydroxyalkyl radical, a C₂-C₄ polyhydroxyalkyl radical, a C₁-C₄ monohydroxyalkoxy radical or a C₂-C₄ polyhydroxyalkoxy radical;
R′₁ and R′₂, which may be identical or different, represent a hydrogen atom or a halogen atom chosen from chlorine, bromine, iodine and fluorine, a C₁-C₄ alkyl radical, a C₁-C₄ monohydroxyalkyl radical, a C₂-C₄ polyhydroxyalkyl radical, a C₁-C₄ alkoxy radical, a C₁-C₄ monohydroxyalkoxy radical or a C₂-C₄ polyhydroxyalkoxy radical;

R′₃ and R′₄, which may be identical or different, represent a hydrogen atom, a C₁-C₄ alkyl radical, a C₁-C₄ monohydroxyalkyl radical, a C₂-C₄ polyhydroxyalkyl radical or a C₁-C₄ monoaminoalkyl radical;

it being understood that:

at least one of the radicals R₁ to R₄ is other than a hydrogen atom;
at least one of the radicals R₁, R₃ and R₄ represents a hydrogen atom;
at least one of the radicals R′₁ and R′₂ represents a halogen atom;
and when R₁, R₂ and R₃ simultaneously represent a hydrogen atom, then R₄ is other than a methyl radical.

The dye composition in accordance with the invention gives intense, highly chromatic colorations which have excellent properties of resistance with respect both to atmospheric agents such as light and bad weather, and to perspiration and the various treatments to which the hair may be subjected. These properties are particularly noteworthy, in particular as regards the selectivity and the resistance of the colorations towards the action of permanent-waving.

A subject of the invention is also a process for the oxidation dyeing of keratin fibres using this dye composition.

Among the substituted meta-aminophenols of formulae (I) and (Ia) above which may be mentioned more particularly are 5-N-(β-hydroxyethyl)amino-2-methylphenol, 3-amino-2-chloro-6-methylphenol, 5-amino-2-methoxyphenol, 5-amino-2-(β-hydroxyethyloxy)phenol, 5-N-(β-hydroxyethyl)amino-4-methoxy-2-methylphenol, 5-amino-4-methoxy-2-methylphenol, 5-amino-4-chloro-2-methylphenol, 5-amino-2,4-dimethoxyphenol, 5-(γ-hydroxypropylamino)-2-methylphenol, 3-amino-6-chlorophenol, 3-amino-6-bromophenol, 3-(β-aminoethyl)amino-6-chlorophenol and 3-(β-hydroxyethyl)amino-6-chlorophenol, and the addition salts thereof with an acid.

The nature of the oxidation base(s) used in the ready-to-use dye composition is not critical. They can be chosen in particular from para-phenylenediamines, double bases, para-aminophenols, ortho-aminophenols and heterocyclic oxidation bases.

Among the para-phenylenediamines which can be used as oxidation base in the dye compositions in accordance with the invention, mention may be made in particular of the compounds of formula (II) below, and the addition salts thereof with an acid: 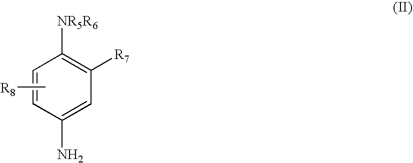
in which:
R₅ represents a hydrogen atom, a C₁-C₄ alkyl radical, a C₁-C₄ monohydroxyalkyl radical, a C₂-C₄ polyhydroxyalkyl radical, a (C₁-C₄)alkoxy(C₁-C₄)alkyl radical or a C₁-C₄ alkyl radical substituted with a nitrogenous, phenyl or 4′-aminophenyl group;
R₆ represents a hydrogen atom, a C₁-C₄ alkyl radical, a C₁-C₄ monohydroxyalkyl radical, a C₂-C₄ polyhydroxyalkyl radical, a (C₁-C₄)alkoxy(C₁-C₄)alkyl radical or a C₁-C₄ alkyl radical substituted with a nitrogenous group;
R₇ represents a hydrogen atom, a halogen atom such as a chlorine, bromine, iodine or fluorine atom, a C₁-C₄ alkyl radical, a C₁-C₄ monohydroxyalkyl radical, a C₁-C₄ hydroxyalkoxy radical, a C₁-C₄ acetylaminoalkoxy radical, a C₁-C₄ mesylaminoalkoxy radical or a C₁-C₄ carbamoylaminoalkoxy radical;
R₈ represents a hydrogen or halogen atom or a C₁-C₄ alkyl radical.

Among the nitrogenous groups of formula (II) above which may be mentioned in particular are amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

Among the para-phenylenediamines of formula (II) above, mention may be made more particularly of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylene-diamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylamino-ethyloxy-para-phenylenediamine and N-(β-methoxyethyl)-para-phenylenediamine, and the addition salts thereof with an acid.

Among the para-phenylenediamines of formula (II) above which are most particularly preferred are para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid.

According to the invention, the expression “double bases” means compounds comprising at least two aromatic nuclei bearing amino and/or hydroxyl groups.

Among the double bases which can be used as oxidation bases in the dye compositions in accordance with the invention, mention may be made in particular of the compounds corresponding to formula (III) below, and the addition salts thereof with an acid: 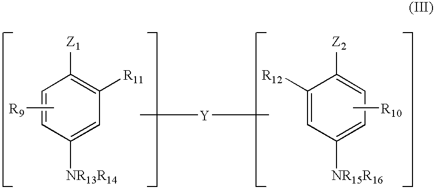
in which:
Z₁ and Z₂, which may be identical or different, represent a hydroxyl or —NH₂ radical which can be substituted with a C₁-C₄ alkyl radical or with a linker arm Y;
the linker arm Y represents a linear or branched alkylene chain comprising from 1 to 14 carbon atoms, which can be interrupted or terminated with one or more nitrogenous groups and/or with one or more hetero atoms such as oxygen, sulphur or nitrogen atoms, and optionally substituted with one or more hydroxyl or C₁-C₆ alkoxy radicals;
R₉ and R₁₀ represent a hydrogen or halogen atom, a C₁-C₄ alkyl radical, a C₁-C₄ monohydroxyalkyl radical, a C₂-C₄ polyhydroxyalkyl radical, a C₁-C₄ aminoalkyl radical or a linker arm Y;
R₁₁, R₁₂, R₁₃, R₁₄, R₁₅ and R₁₆, which may be identical or different,

represent a hydrogen atom, a linker arm Y or a C₁-C₄ alkyl radical; it being understood that the compounds of formula (III) comprise only one linker arm Y per molecule.

Among the nitrogenous groups of formula (III) above which may be mentioned in particular are amino, mono(C₁-C₄)alkylamino, di(C₁-C₄)alkylamino, tri(C₁-C₄)alkylamino, monohydroxy(C₁-C₄)alkylamino, imidazolinium and ammonium radicals.

Among the double bases of formula (III) above which may be mentioned more particularly are N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetra-methylenediamine, N,N′-bis(ethyl)-N,N′-bis(4′-amino-3′-methylphenyl)ethylenediamine and 1,8-bis(2,5-diaminophenoxy)-3,5-dioxaoctane, and the addition salts thereof with an acid.

Among these double bases of formula (III), N,N′-bis(β-hydroxyethyl)-N,N′-bis(4′-aminophenyl)-1,3-diaminopropanol and 1,8-bis(2,5-diaminophenoxy)-3,5-dioxaoctane, or one of the addition salts thereof with an acid, are particularly preferred.

Among the para-aminophenols which can be used as oxidation bases in the dye compositions in accordance with the invention, mention may be made in particular of the compounds of formula (IV) below, and the addition salts thereof with an acid: 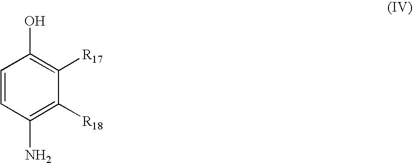
in which:
R₁₇ represents a hydrogen or halogen atom, a C₁-C₄ alkyl radical, a C₁-C₄ monohydroxyalkyl radical, a (C₁-C₄)alkoxy(C₁-C₄)alkyl radical, a C₁-C₄ aminoalkyl radical or a hydroxy(C₁-C₄)alkylamino(C₁-C₄)alkyl radical,
R₁₈ represents a hydrogen or halogen atom, a C₁-C₄ alkyl radical, a C₁-C₄ monohydroxyalkyl radical, a C₂-C₄ polyhydroxyalkyl radical, a C₁-C₄ aminoalkyl radical, a cyano(C₁-C₄)alkyl radical or a (C₁-C₄)alkoxy(C₁-C₄)alkyl radical,
   it being understood that at least one of the radicals R₁₇ and R₁₈ represents a hydrogen atom.

Among the para-aminophenols of formula (IV) above which may be mentioned more particularly are para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols which can be used as oxidation bases in the dye compositions in accordance with the invention, mention may be made more particularly of 2-aminophenol, 2-amino-5-methylphenol, 2-amino-6-methylphenol and 5-acetamido-2-aminophenol, and the addition salts thereof with an acid.

Among the heterocyclic bases which can be used as oxidation bases in the dye compositions in accordance with the invention, mention may be made more particularly of pyridine derivatives, pyrimidine derivatives and pyrazole derivatives, and the addition salts thereof with an acid.

Among the pyridine derivatives which may be mentioned more particularly are the compounds described, for example, in patents GB 1,026,978 and GB 1,153,196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine and 3,4-diaminopyridine, and the addition salts thereof with an acid.

Among the pyrimidine derivatives, mention may be made more particularly of the compounds described, for example, in German patent DE 2,359,399 or Japanese patents JP 88-169,571 and JP 91-10659 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine, as well as pyrazolopyrimidine derivatives such as those mentioned in patent application FR-A-2,750,048 and among which mention may be made of pyrazolo[1,5-a]pyrimidine-3,7-diamine, 2,5-dimethylpyrazolo[1,5-a]pyrimidine-3,7-diamine, pyrazolo[1,5-a]pyrimidine-3,5-diamine, 2,7-dimethylpyrazolo[1,5-a]pyrimidine-3,5-diamine, 3-aminopyrazolo[1,5-a]pyrimidin-7-ol, 3-aminopyrazolo[1,5-a]pyrimidin-5-ol, 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine and 2,5-N-7,N-7-tetramethylpyrazolo[1,5-a]pyrimidine-3,7-diamine, and the addition salts thereof and the tautomers thereof, when a tautomeric equilibrium exists, and the addition salts thereof with an acid.

Among the pyrazole derivatives, mention may be made more particularly of the compounds described in patents or patent applications DE 3,843,892, DE 4,133,957, WO 94/08969, WO 94/08970, FR-A-2,733,749 and DE 195 43 988 such as 4,5-diamino-1-methylpyrazole, 3,4-diaminopyrazole, 4,5-diamino-1-(4′-chloro-benzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(b-hydroxyethyl)-3-methylpyrazole, 4,5-diamino-1-ethyl-3-methylpyrazole, 4,5-diamino-1ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(b-hydroxyethyl)amino-1-methylpyrazole, and the addition salts thereof with an acid.

The oxidation base(s) preferably represent(s) from 0.0005 to 12% by weight approximately relative to the total weight of the dye composition in accordance with the invention, and even more preferably from 0.005 to 6% by weight approximately relative to this weight.

The 1,3-bis(β-hydroxyethyl)amino-2-methylbenzene and/or the addition salt(s) thereof with an acid, which are used as first coupler according to the invention, preferably represent(s) from 0.001 to 10% by weight approximately relative to the total weight of the dye composition and even more preferably from 0.01 to 5% by weight approximately relative to this weight.

The substituted meta-aminophenol(s) of formulae (I) and/or (Ia) in accordance with the invention preferably represents from 0.0001 to 10% by weight approximately relative to the total weight of the dye composition and even more preferably from 0.005 to 5% by weight approximately relative to this weight.

The dye composition in accordance with the invention can also contain one or more additional couplers other than 1,3-bis(β-hydroxyethyl)amino-2-methylbenzene, substituted meta-aminophenols of formulae (I) and (Ia) and the addition salts thereof with an acid, and/or one or more direct dyes, in particular to modify the shades or to enrich them with glints.

Among the couplers which may additionally be present in the dye composition in accordance with the invention, mention may be made in particular of meta-phenylenediamines, meta-diphenols and heterocyclic couplers, and the addition salts thereof with an acid.

When they are present, these additional couplers preferably represent from 0.0001 to 10% by weight approximately relative to the total weight of the dye composition, and even more preferably from 0.005 to 5% by weight approximately relative to this weight.

In general, the addition salts with an acid which can be used in the context of the dye compositions of the invention (oxidation bases and couplers) are chosen in particular from the hydrochlorides, hydrobromides, sulphates, tartrates, lactates and acetates.

The medium which is suitable for dyeing (or support) for the dye composition in accordance with the invention generally consists of water or a mixture of water and at least one organic solvent to dissolve the compounds which would not be sufficiently soluble in water. Organic solvents which may be mentioned, for example, are C₁-C₄ alkanols, such as ethanol and isopropanol.

The solvents can be present in proportions preferably of between 1 and 40% by weight approximately relative to the total weight of the dye composition, and even more preferably between 5 and 30% by weight approximately.

The pH of the dye composition in accordance with the invention is generally between 3 and 12 approximately, and preferably between 5 and 12 approximately. It can be adjusted to the desired value using acidifying or basifying agents usually used for dyeing keratin fibres.

Among the acidifying agents which may be mentioned, for example, are inorganic or organic acids such as hydrochloric acid, orthophosphoric acid, sulphuric acid, carboxylic acids such as acetic acid, tartaric acid, citric acid and lactic acid, and sulphonic acids.

Among the basifying agents which may be mentioned, for example, are aqueous ammonia, alkaline carbonates, alkanolamines such as mono-, di- and triethanolamine, 2-methyl-2-aminopropanol and derivatives thereof, sodium hydroxide, potassium hydroxide and the compounds of formula (V) below: 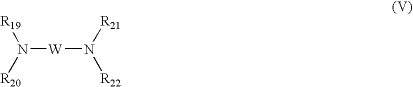
in which W is a propylene residue optionally substituted with a hydroxyl group or a C₁-C₄ alkyl radical; R₁₉, R₂₀, R₂, and R₂₂, which may be identical or different, represent a hydrogen atom or a C₁-C₄ alkyl or C₁-C₄ hydroxyalkyl radical.

The dye composition in accordance with the invention can also contain various adjuvants conventionally used in compositions for dyeing the hair.

Needless to say, a person skilled in the art will take care to select this or these optional additional compounds such that the advantageous properties intrinsically associated with the dye composition in accordance with the invention are not, or are not substantially, adversely affected by the addition(s) envisaged.

The dye composition in accordance with the invention can be in various forms, such as in the form of liquids, creams or gels, which are optionally pressurized, or in any other form which is suitable for dyeing keratin fibres, and in particular human hair.

A subject of the invention is also a process for dyeing keratin fibres, and in particular human keratin fibres such as the hair, using the dye composition as defined above.

According to this process, the dye composition as defined above is applied to the fibres, the colour being developed at acidic, neutral or alkaline pH using an oxidizing agent which is added to the dye composition just at the time of use, or which is present in an oxidizing composition that is applied simultaneously or sequentially in a separate manner.

According to one particularly preferred embodiment of the dyeing process according to the invention, the dye composition described above is mixed, at the time of use, with an oxidizing composition comprising, in a medium which is suitable for dyeing, at least one oxidizing agent which is present in an amount which is sufficient to develop a coloration. The mixture obtained is then applied to the keratin fibres and is left to stand for 3 to 50 minutes approximately, preferably 5 to 30 minutes approximately, after which the fibres are rinsed, washed with shampoo, rinsed again and dried.

The oxidizing agent present in the oxidizing composition as defined above can be chosen from the oxidizing agents conventionally used for the oxidation dyeing of keratin fibres, and among which mention may be made of hydrogen peroxide, urea peroxide, alkali metal bromates, persalts such as perborates and persulphates, peracids, enzymes such as 2-electron oxidoreductases, peroxidases and lactases. Hydrogen peroxide is particularly preferred.

The pH of the oxidizing composition containing the oxidizing agent as defined above is such that, after mixing with the dye composition, the pH of the resulting composition applied to the keratin fibres preferably ranges between 3 and 12 approximately and even more preferably between 5 and 11. It is adjusted to the desired value using acidifying or basifying agents usually used for dyeing keratin fibres and as defined above.

The oxidizing composition as defined above can also contain various adjuvants conventionally used in compositions for dyeing the hair and as defined above.

The composition which is finally applied to the keratin fibres can be in various forms, such as in the form of liquids, creams or gels, or in any other form which is suitable for dyeing keratin fibres, and in particular human hair.

Another subject of the invention is a multi-compartment dyeing device or multi-compartment dyeing “kit” or any other multi-compartment packaging system, a first compartment of which contains the dye composition as defined above and a second compartment of which contains the oxidizing composition as defined above. These devices can be equipped with a means for applying the desired mixture to the hair, such as the devices described in patent FR-2,586,913 in the name of the Applicant.

The examples which follow are intended to illustrate the invention without thereby limiting its scope.

### EXAMPLES

### Examples 1 and 2 of Dyeing in Alkaline Medium

The dye compositions below in accordance with the invention were prepared (contents in grams):

EXAMPLE

| | 1 | 2 |
|---|---|---|
| para-Phenylenediamine (oxidation base) | 0.22 | — |
| para-Aminophenol (oxidation base) | — | 0.44 |
| 1,3-Bis(β-hydroxyethyl)amino-2-methylbenzene | 0.21 | 0.21 |
| (first coupler) | | |
| 5-N-(β-Hydroxyethyl)amino-2-methylphenol | 0.17 | — |
| (coupler of formula (I)) | | |
| 3-Amino-2-chloro-6-methylphenol (coupler of | — | 0.32 |
| formula (Ia)) | | |
| Common dye support | (*) | (*) |
| Demineralized water qs | 100 g | 100 g |

| | | |
|---|---|---|
| (*): Common dye support No. 1: | | |
| 96° ethanol | 18 | g |
| Sodium metabisulphite as an aqueous 35% | 0.68 | g |
| solution | | |
| Pentasodium salt of diethylenetriamine | 1.1 | g |
| pentaacetic acid | | |
| Aqueous ammonia containing 20% NH₃ | 10 | g |

At the time of use, each of the dye compositions described above was mixed with an equivalent amount by weight of 20-volumes hydrogen peroxide (6% by weight) having a pH of about 3.

Each resulting mixture had a pH of about 10±0.2 and was applied for 30 minutes to locks of permanent-waved grey hair containing 90% white hairs.

The hair was then rinsed with water, washed with a standard shampoo, rinsed again and then dried.

The hair was dyed in the shades given in the table below:

| EXAMPLE | SHADE OBTAINED |
|---|---|
| 1 | Iridescent dark puple |
| 2 | Coppery red |

### Example 3 of Dyeing in Acidic Medium

The dye composition below in accordance with the invention was prepared:

| | | |
|---|---|---|
| para-Phenylenediamine (oxidation base) | 0.21 | g |
| 1,3-Bis(β-Hydroxyethyl)amino-2-methyl- | 0.21 | g |
| benzene (first coupler) | | |
| 5-N-(β-Hydroxyethyl)amino-2-methyl- | 0.17 | g |
| phenol (coupler of formula (I)) | | |
| 96° ethanol | 18 | g |
| Sodium metabisulphite as an aqueous 35% | 0.68 | g |
| solution | | |
| Pentasodium salt of diethylenetriamine- | 1.1 | g |
| pentaacetic acid | | |
| K₂HPO₄/KH₂PO₄ (1.5 M/1M) buffer | 10 | g |
| Demineralized water qs | 100 | g |

At the time of use, the dye composition described above was mixed with an equivalent amount by weight of 20-volumes hydrogen peroxide (6% by weight) having a pH of about 3.

The resulting mixture had a pH of about 6.8±0.2, and was applied for 30 minutes to locks of permanent-waved grey hair containing 90% white hairs.

The hair was then rinsed with water, washed with a standard shampoo, rinsed again and then dried.

The hair was dyed in an iridescent violet shade.

### Comparative Examples 4 and 5

The dye compositions below were prepared (contents in grams):

| | EXAMPLE | |
|---|---|---|
| | 4 | 5(**) |
| para-Aminophenol (oxidation base) | 0.327 | 0.327 |
| 1,3-Bis(β-Hydroxyethyl)amino-2-methylbenzene | 0.315 | 0.315 |
| (first coupler) | | |
| 2-Methyl-5-N-(β-hydroxyethyl)aminophenol | 0.2505 | — |
| (coupler of formula (I): 1.5 × 10⁻³ mol) | | |
| 2-Methyl-5-aminophenol (coupler not forming part | — | 0.1845 |
| of the invention: 1.5 × 10⁻³ mol) | | |
| Common dye support No. 2 | (***) | (***) |
| Demineralized water qs | 100 g | 100 g |

| (**): comparative example not forming part of the | | |
|---|---|---|
| invention | | |
| It is important to note that each of the dye compositions | | |
| of Examples 5 and 6 above contains the same molar | | |
| amount of additional coupler, i.e. 1.5 × 10⁻³ mol. | | |
| (***): Common dye support No. 2: | | |
| Oleyl alcohol polyglycerolated with 2 mol of glycerol | 4.0 | g |
| Oleyl alcohol polyglycerolated with 4 mol of glycerol, | 5.69 | g A.M. |
| containing 78% active material (A.M.) | | |
| Oleic acid | 3.0 | g |
| Oleylamine containing 2 mol of ethylene oxide, sold | 7.0 | g |
| under the trade name Ethomeen O12 ® by the | | |
| company Akzo | | |
| Diethylaminopropyl laurylamino-succinamate, | 3.0 | g A.M. |
| sodium salt, containing 55% A.M. | | |
| Oleyl alcohol | 5.0 | g |
| Oleic acid diethanolamide | 12.0 | g |
| Propylene glycol | 3.5 | g |
| Ethyl alcohol | 7.0 | g |
| Dipropylene glycol | 0.5 | g |
| Propylene glycol monomethyl ether | 9.0 | g |
| Sodium metabisulphite as an aqueous solution | 0.455 | g A.M. |
| containing 35% A.M. | | |
| Ammonium acetate | 0.8 | g |
| Antioxidant, sequestering agent | qs | |
| Fragrance, preserving agent | qs | |
| Aqueous ammonia containing 20% NH₃ | 10.0 | g |

At the time of use, each of the dye compositions described above was mixed with an equivalent amount by weight of 20-volumes hydrogen peroxide (6% by weight) having a pH of about 3.

Each resulting mixture had a pH of about 10±0.2, and was applied for 30 minutes to locks of permanent-waved grey hair containing 90% white hairs.

The hair was then rinsed with water, washed with a standard shampoo, rinsed again and then dried.

The colour of the locks was evaluated in the Munsell system using a Minolta CM 2002® spectrophotometer.

A test of resistance to permanent-waving was carried out on the locks of hair thus dyed.

To do this, the locks of hair were impregnated for 10 minutes with a reducing solution at a rate of 2 g of the reducing solution below per lock of 1 g:

Reducing Solution:

| Thioglycolic acid | 6.7 | g |
|---|---|---|
| Diammonium dithioglycolate at 48% in water | 5.0 | g |
| Basifying agent qs | pH = 7.9 | |
| Demineralized water qs | 100.0 | g |

After rinsing, the locks of hair were immersed for 5 minutes in an oxidizing solution (consisting of an 8-volumes hydrogen peroxide solution of pH 3) at a rate of 2 g of oxidizing solution per lock of 1 g.

The locks were then rinsed with water and then dried for 1 hour at 60° C.

The colour of the locks of hair was then evaluated again in the Munsell system using a Minolta CM 2002® spectrophotometer.

The difference between the colour of the lock before permanent-waving and the colour of the lock after permanent-waving was calculated by applying the Nickerson formula:
ΔE=0.4CoΔH+6ΔV+3ΔC
as described, for example, in “Couleur, Industrie et Technique [Colour, Industry and Technology]”; pages 14-17; Vol. No. 5; 1978.

In this formula, ΔE represents the difference in colour between two locks, ΔH, ΔV and ΔC represent the variation in the absolute value of the parameters H, V and C, and Co represents the purity of the lock relative to which it is desired to evaluate the colour difference (before the test of resistance to permanent-waving).

The degradation of the colour (ΔE) is proportionately greater the higher the value indicated.

The results are given in Table I below:

| | Colour of | | | | | |
|---|---|---|---|---|---|---|
| | the hair | Colour of the | | | | |
| | before | hair after | Degradation of the | | | |
| | permanent- | permanent- | colour | | | |
| EXAMPLE | waving | waving | ΔH | ΔV | ΔC | ΔE |
| 4 | 3.8 R 3.4/5.0 | 3.7 R 4.0/3.9 | 0.1 | 0.6 | 1.1 | 7.1 |
| 5(**) | 4.7 R 3.2/4.8 | 3.3 R 4.1/3.5 | 1.4 | 0.9 | 1.3 | 12.0 |

(**)Example not forming part of the invention.

These results show that the dye composition of Example 4 in accordance with the invention, containing a combination of an oxidation base (para-aminophenol), 1,3-bis(β-hydroxyethyl)amino-2-methyl benzene as first coupler, and 2-methyl-5-N-(β-hydroxyethyl)aminophenol as additional coupler in accordance with formula (I), gives a coloration which withstands the action of permanent-waving much better than the coloration obtained using the composition of Example 5, which does not form part of the invention since, instead of 2-methyl-5-N-(β-hydroxyethyl)aminophenol, it contains 2-methyl-5-aminophenol as described, for example, in patent application FR 2,681,860.

### Comparative Examples 6 and 7

The dye compositions below were prepared (contents in grams):

| | EXAMPLE | |
|---|---|---|
| | 6 | 7(**) |
| para-Aminophenol (oxidation base) | 0.327 | 0.327 |
| 1,3-Bis(β-Hydroxyethyl)amino-2-methylbenzene | 0.315 | 0.315 |
| (first coupler) | | |
| 2-Methyl-5-N-(β-hydroxyethyl)aminophenol | 0.2505 | — |
| (coupler of formula (I): 1.5 × 10⁻³ mol) | | |
| meta-Aminophenol (coupler not forming part of | — | 0.1635 |
| the invention: 1.5 × 10⁻³ mol) | | |
| Common dye support No. 2 | (***) | (***) |
| Demineralized water qs | 100 g | 100 g |

(**): comparative example not forming part of the invention It is important to note that each of the dye compositions of Examples 5 and 6 above contains the same molar amount of additional coupler, i.e. 1.5 × 10⁻³ mol.
(***): Common dye support No. 2:

This is identical to the one used for Comparative Examples 4 and 5 above.

At the time of use, each of the dye compositions described above was mixed with an equivalent amount by weight of 20-volumes hydrogen peroxide (6% by weight) having a pH of about 3.

Each resulting mixture had a pH of about 10±0.2, and was applied for 30 minutes, on the one hand, to locks of natural grey hair containing 90% white hairs, and, on the other hand, to locks of permanent-waved grey hair containing 90% white hairs.

The hair was then rinsed with water, washed with a standard shampoo, rinsed again and then dried.

The colour of the locks was evaluated in the Munsell system using a Minolta CM 2002® spectrophotometer.

The difference in colour between the locks of natural dyed hair and the locks of permanently-waved dyed hair corresponding to the selectivity of the coloration was calculated by applying the Nickerson formula:
ΔE=0.4CₒdH+6dV+3dC
as described, for example, in “Journal of the Optical Society of America”, Vol. 34, No. 9, September 1944, pages 550-570.

In this equation, ΔE represents the difference in colour between two locks (in the present case the selectivity of the coloration), dH, dV and dC represent the variation in the absolute value of the three parameters H, V and C, and Co represents the saturation of the lock relative to which it is desired to evaluate the difference in colour.

The larger the value of ΔE, the greater the difference in colour between the two locks, and, in the present case, the greater the selectivity of the coloration.

The results are given in the table below:

| | | Colour on | Selectivity of the | | | |
|---|---|---|---|---|---|---|
| | Colour on | permanently- | coloration | | | |
| EXAMPLE | natural hair | waved hair | dH | dV | dC | ΔE |
| 6 | 5.1 R 3.8/3.7 | 3.8 R 3.4/5.0 | 1.3 | 0.4 | 1.3 | 8.2 |
| 7(**) | 2.6 R 3.8/3.3 | 8.3 RP 3.2/4.9 | 4.3 | 0.6 | 1.6 | 14.1 |

These results show that the dye composition of Example 6 in accordance with the invention, containing a combination of an oxidation base (para-aminophenol), 1,3-bis(β-hydroxyethyl)amino-2-methylbenzene as first coupler, and 2-methyl-5-N-(β-hydroxyethyl)aminophenol as additional coupler in accordance with formula (I), gives a coloration which is markedly less selective (more uniform) than the coloration obtained using the composition of Example 7, which does not form part of the invention since, instead of 2-methyl-5-N-(β-hydroxyethyl)aminophenol, it contains meta-aminophenol as described, for example, in patent application FR 2,681,860.

1-32. (canceled)

33. A composition for the oxidation dyeing of keratin fibres comprising, in a medium which is suitable for dyeing:
(a) at least one oxidation base,
(b) and at least two couplers, wherein
the first coupler is chosen from 1,3-bis(β-hydroxyethyl)amino-2-methylbenzene and an addition salt thereof with an acid, and
the second coupler is chosen from 3-amino-2-chloro-6-methylphenol, 5-amino-4-chloro-2-methylphenol, and the addition salts thereof with an acid.

34. The composition according to claim 34, wherein the oxidation base is chosen from a para-phenylenediamine, a double base, a para-aminophenol, an ortho-aminophenol, and a heterocyclic oxidation base.

35. The composition according to claim 34, wherein the para-phenylenediamine corresponds to formula (II), and an addition salt thereof with an acid: 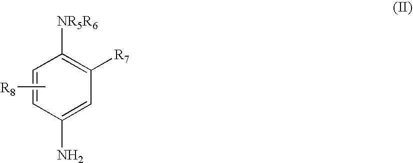
in which:
R₅ is chosen from hydrogen, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals, C₁-C₄ alkyl radicals substituted with a nitrogenous group, C₁-C₄ alkyl radicals substituted with a phenyl group, and C₁-C₄ alkyl radicals substituted with a 4′-aminophenyl group;
R₆ is chosen from hydrogen, C₁-C₄ alkyl radicals, C₁-C₄ monohydroxyalkyl radicals, C₂-C₄ polyhydroxyalkyl radicals, (C₁-C₄)alkoxy(C₁-C₄)alkyl radicals, and C₁-C₄ alkyl radicals substituted with a nitrogenous group;
R₇ is chosen from hydrogen, halogen, C₁-C₄ alkyl radicals, C₁-C₄ monohydroxyalkyl radicals, C₁-C₄ hydroxyalkoxy radicals, C₁-C₄ acetylamino-alkoxy radicals, C₁-C₄ mesylaminoalkoxy radicals, and C₁-C₄ carbamoylaminoalkoxy radicals; and
R₈ is chosen from hydrogen, halogen, and C₁-C₄ alkyl radicals.

36. The composition according to claim 35, wherein R₇ is a halogen chosen from chlorine, bromine, iodine, and fluorine.

37. The composition according to claim 35, wherein the para-phenylenediamine of formula (II) is chosen from para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxy-ethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, and the addition salts thereof with an acid.

38. The composition according to claim 34, wherein the double base corresponds to formula (III), and an addition salt thereof with an acid: 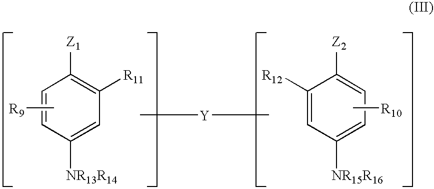
in which:
Z₁ and Z₂, which may be identical or different, are chosen from a hydroxyl radical or an —NH₂ radicals, which can be substituted with C₁-C₄ alkyl radicals or with a linker arm Y;
the linker arm Y is chosen from a linear or branched alkylene chain comprising from 1 to 14 carbon atoms, which can be interrupted or terminated with at least one nitrogenous group, with at least one hetero atom, or with a mixture thereof;
R₉ and R₁₀ are chosen from hydrogen, halogen atoms, C₁-C₄ alkyl radicals, C₁-C₄ monohydroxyalkyl radicals, C₂-C₄ polyhydroxyalkyl radicals, C₁-C₄ aminoalkyl radicals and a linker arm Y;
R₁₁, R₁₂, R₁₃, R₁₄, R₁₅ and R₁₆, which may be identical or different, are chosen from hydrogen, a linker arm Y and C₁-C₄ alkyl radicals;
provided that the compound of formula (III) comprises only one linker arm Y per molecule.

39. The composition according to claim 38, wherein the hetero atom that can interrupt or terminate the linker arm Y is chosen from oxygen, sulphur or nitrogen.

40. The composition according to claim 38, wherein the linker arm Y is substituted with at least one group chosen from a hydroxyl radical and C₁-C₆ alkoxy radicals.

41. The composition according to claim 38, wherein the double base of formula (III) is chosen from N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetra-methylenediamine, N,N′-bis(ethyl)-N,N′-bis(4′-amino-3′-methylphenyl)ethylenediamine, 1,8-bis(2,5-diaminophenoxy)-3,5-dioxaoctane, and the addition salts thereof with an acid.

42. The composition according to claim 34, wherein the para-aminophenol corresponds to formula (IV), and an addition salt thereof with an acid: 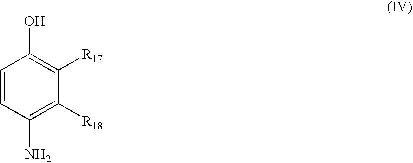
in which:
R₁₇ is chosen from hydrogen, halogen atoms, C₁-C₄ alkyl radicals, C₁-C₄ monohydroxyalkyl radicals, (C₁-C₄)alkoxy(C₁-C₄)alkyl radicals, C₁-C₄ aminoalkyl radicals and hydroxy(C₁-C₄)alkylamino(C₁-C₄)alkyl radicals; and
R₁₈ is chosen from hydrogen, halogen atoms, C₁-C₄ alkyl radicals, C₁-C₄ monohydroxyalkyl radicals, C₂-C₄ polyhydroxyalkyl radicals, C₁-C₄ aminoalkyl radicals, cyano(C₁-C₄)alkyl radicals, and (C₁-C₄)alkoxy(C₁-C₄)alkyl radicals,
provided that at least one of the radicals R₁₇ and R₁₈ is hydrogen.

43. The composition according to claim 42, wherein the para-aminophenol of formula (IV) is chosen from para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethyl-aminomethyl)phenol, 4-amino-2-fluorophenol, and the addition salts thereof with an acid.

44. The composition according to claim 34, wherein the ortho-aminophenol is chosen from 2-aminophenol, 2-amino-5-methylphenol, 2-amino-6-methylphenol, 5-acetamido-2-aminophenol, and an addition salt thereof with an acid.

45. The composition according to claim 34, wherein the heterocyclic oxidation base is chosen from pyridine derivatives, pyrimidine derivatives, pyrazole derivatives, and the addition salts thereof with an acid.

46. The composition according to claim 34, wherein the oxidation base is present in said composition in an amount ranging from 0.0005 to 12% by weight relative to the total weight of the composition.

47. The composition according to claim 46, wherein the oxidation base is present in said composition in an amount ranging from 0.005 to 6% by weight relative to the total weight of the composition.

48. The composition according claim 33, wherein the first coupler is present in said composition in an amount ranging from 0.001 to 10% by weight relative to the total weight of the composition.

49. The composition according to claim 48, wherein the first coupler is present in said composition in an amount ranging from 0.01 to 5% by weight relative to the total weight of the composition.

50. The composition according to claim 33, wherein the second coupler is present in said composition in an amount ranging from 0.0001 to 10% by weight relative to the total weight of the composition.

51. The composition according to claim 50, wherein the second coupler is present in said composition in an amount of from 0.005 to 5% by weight relative to the total weight of the composition.

52. The composition according to claim 33, further comprising at least one additional coupler that is different from the first coupler and the second coupler, and at least one direct dye.

53. The composition according to claim 33, further comprising at least one direct dye.

54. The composition according to claim 33, further comprising at least one additional coupler that is different from the first coupler and the second coupler.

55. The composition according to claim 33, wherein each addition salt with an acid is independently chosen from a hydrochloride, a hydrobromide, a sulphate, a tartrate, a lactate, and an acetate.

56. The composition according to claim 33, wherein the keratin fibres are human keratin fibres.

57. The composition according to claim 56, wherein the human keratin fibres are hair.

58. A process for dyeing keratin fibres comprising the steps of 1) applying to said fibres at least one dye composition, and 2) developing a color at acidic, neutral or alkaline pH using an oxidizing agent which is added to the dye composition at the time of use, or which is present in an oxidizing composition that is applied simultaneously with the dye composition or sequentially after application of the dye composition, said at least one dye composition comprising, in a medium which is suitable for dyeing:
(a) at least one oxidation base,
(b) and at least two couplers, wherein
the first coupler is chosen from 1,3-bis(β-hydroxyethyl)amino-2-methylbenzene and an addition salt thereof with an acid, and
the second coupler is chosen from 3-amino-2-chloro-6-methylphenol, 5 5-amino-4-chloro-2-methylphenol, and the addition salts thereof with an acid.

59. The process according to claim 58, wherein the oxidizing agent present in the oxidizing composition is chosen from hydrogen peroxide, urea peroxide, an alkali metal bromate, a persalt, a peracid, and an enzyme.

60. The process according to claim 59, wherein the persalt is chosen from perborates, percarbonates, and persulphates.

61. The process according to claim 58, wherein the keratin fibers are human keratin fibres.

62. The process according to claim 61, wherein the human keratin fibers are hair.

63. A multi-compartment dyeing kit comprising
a first compartment that contains a dye composition for the oxidation dyeing of keratin fibers, and a second compartment that contains an oxidizing composition, said dye composition comprising, in a medium which is suitable for dyeing:
(a) at least one oxidation base,
(b) and at least two couplers, wherein
the first coupler is chosen from 1,3-bis(β-hydroxyethyl)amino-2-methylbenzene and an addition salt thereof with an acid, and
the second coupler is chosen from 3-amino-2-chloro-6-methylphenol, 5-amino-4-chloro-2-methylphenol, and the addition salts thereof with an acid.
